(19) United States
(12) Patent Application Publication
(10) Pub. No.: US 2025/0274116 A1
(43) Pub. Date: Aug. 28, 2025

(54) OVER-CURRENT PROTECTION FOR A POWER SUPPLY

(71) Applicant: Advanced Energy Industries, Inc., Denver, CO (US)

(72) Inventors: Robert Ryan, Cork (IE); Mark Corpuz, Cork (IE); Andrejs Gasilovs, Midleton (IE); Jack William Bermingham, Kinsale (IE)

(21) Appl. No.: 18/590,299

(22) Filed: Feb. 28, 2024

(51) Int. Cl. H03K 17/082; A61N 1/36; H03K 3/57

(52) U.S. Cl. CPC H03K 17/0822; A61N 1/36002; H03K 3/57; H03K 2217/0027

(57) ABSTRACT

A power supply and associated method are disclosed. The power supply comprises a pulse generator configured to receive power from an electrical source and provide power between a first node and a second node. A switch circuit is coupled to the pulse generator, and a sensor is configured to sense current of the power supply and to generate a signal indicative of the current. Control logic is configured, in response to the signal exceeding a threshold, to control the switch circuit to block forward current from the electrical source while simultaneously allowing reverse current to flow to the electrical source.

### FIELD OF THE DISCLOSURE

This disclosure relates generally to power supplies. More particularly, this disclosure relates to an over-current protection (OCP) to limit the amount of energy that can be delivered by a power supply.

### BACKGROUND

A pulsed electric field (PEF) generator is a type of power supply that is configured to generate suitable output waveshapes for a wide array of applications. Generally, the PEF generator produces short, intense bursts of electric field pulses that are used to apply a high-voltage electric field to various substances or materials for a brief period. PEF generators have many possible applications. They may be used in the food industry to inactivate bacteria, yeasts, and molds in food products, thus extending their shelf life. They may be used in water and wastewater treatment to break down pollutants. They may be used in biotechnology to help transfer genes into cells. These are but a few of many possible applications of PEF generators.

Another example of the possible uses of a PEF generator is in medical applications. In one such application, a PEF generator is deployed in a medical device known as an electroporator for use in cancer treatments. The PEF generator of the electroporator generates pulsed output waveforms with amplitudes ranging from 20V to 3 kV depending on the treatment. The pulsed voltage waveforms generated by the electroporator are applied to cancer tumors to induce the biological phenomenon of electroporation and ultimately induce tumor death. PEF generators of this nature can be rated for extremely high peak power levels as high as 150 kW.

PEF generators often use a high internal energy storage capacitor bank that is applied through a switch network to create a clean pulsed square wave output. The capacitor bank stores substantially more energy than is delivered, which is necessary to produce well shaped square wave output voltages. Where the PEF generator is used in a medical application, such as an electroporator, ensuring patient safety is of the utmost importance. However, given the high available output powers and high internal energy storage of PEF generators, the use of such generators has an inherent risk that the switch network could switch incorrectly or be controlled incorrectly, which could potentially cause the entire stored energy of the capacitor bank to be output from the PEF generator and applied directly to the patient, thereby delivering excessive energy that could cause harm such as thermal damage to the patient's tissue. Other applications of PEF generators beyond medical applications may also create a risk of damage or harm by inadvertent output of excessive energy.

### SUMMARY

One aspect of this disclosure may be characterized as a power supply comprising a pulse generator configured to receive power from an electrical source and provide power between a first node and a second node. The power supply also comprises a switch circuit coupled to the pulse generator, a sensor configured to sense current of the power supply and to generate a signal indicative of the current. The power supply also includes control logic configured, in response to the signal exceeding a threshold, to control the switch circuit to block forward current from the electrical source while simultaneously allowing reverse current to flow to the electrical source.

Another aspect may be characterized as a method comprising providing power to a load with a power supply, sensing current of the power supply, and blocking, in response to the current exceeding a threshold, forward current from an electrical source while simultaneously allowing reverse current to flow to the electrical source.

These and other aspects of this disclosure are depicted in the accompanying drawings and description and will be apparent based thereon.

### BRIEF DESCRIPTION OF THE DRAWINGS

FIG. 1 is a diagram depicting an electrical architecture of a power supply.

FIG. 2 is a graph depicting an example waveform that may be applied by power supplies disclosed herein.

FIG. 3 is a diagram depicting an example environment in which the power supplies disclosed herein may be utilized.

FIG. 4A is a block diagram illustrating an example of overcurrent protection circuitry implemented in a power supply.

FIG. 4B is a flowchart depicting a method that may be traversed in connection with embodiments disclosed herein.

FIG. 5 is a graph depicting a typical pulsed output voltage of power supplies disclosed herein.

FIG. 6 is a collection of graphs depicting an overcurrent event.

FIG. 7 is a depiction of current flow in the power supply of FIG. 4A during normal operation.

FIG. 8 is a depiction current flow in the power supply of FIG. 4A responsive to an overcurrent event.

FIG. 9 is a conceptual block diagram of a computing system that may be utilized in connection with embodiments disclosed herein.

### DETAILED DESCRIPTION

As discussed above, a pulsed electric field (PEF) generator is one type of power supply that produces short, intense bursts of electric field pulses that are used to apply a high-voltage electric field to various substances or materials for a brief period. PEF generators find application in many fields, including without limitation the food industry, water and wastewater treatment, biotechnology, and medicine. For purposes of explaining the PEF generator and over-current protection (OCP) circuit of this disclosure in the context of one possible application, the following description is with reference to a PEF generator as used in a medical device known as an electroporator for treatment of cancer. It should be understood, however, that this disclosure is not so limited, and that the PEF generator and OCP circuit described herein may be advantageously deployed in many other applications.

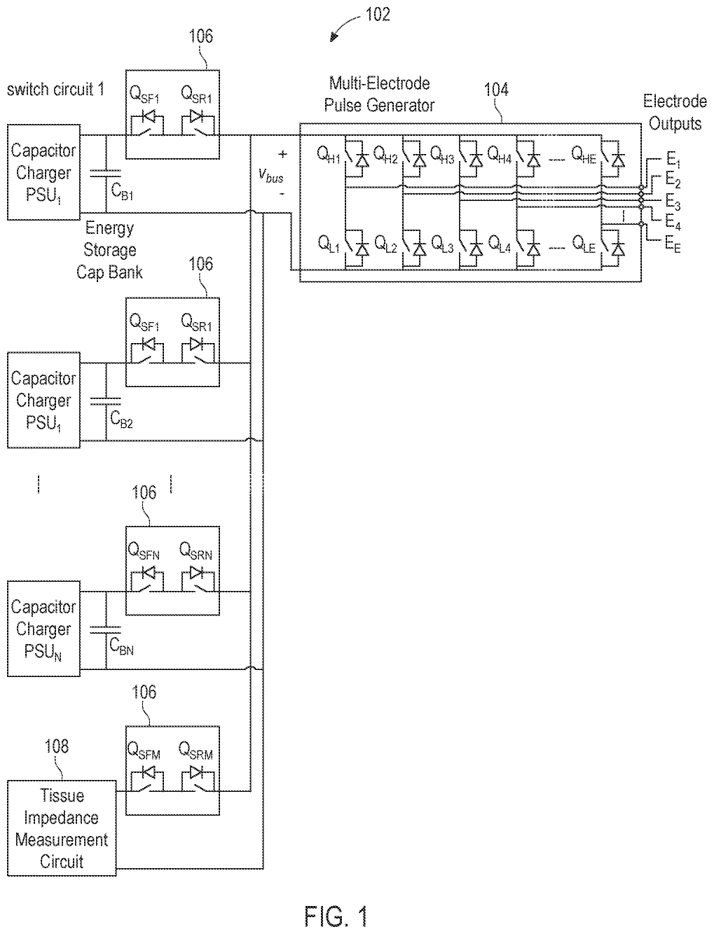

Referring first to FIG. 1, shows the simplified electrical architecture of a power supply 102 that may be used as a pulsed electric-field generator (PEF). For example, as discussed further herein, the power supply 102 may be used to create a pulsed rectangular voltage which may be applied across any two of the electrode outputs E₁-E_{E}. Any of the electrode outputs E₁-E_{E} may be selected to act as the anode, similarly any output may be selected to act as the cathode. The pulsed output is then applied across the anode to the cathode, while the other electrodes remain unactive.

As shown, the power supply 102 may include a pulse generator 104 comprising E half-bridge circuits (where E is one or more), where each half-bridge circuit includes two semiconductor switches. These semiconductor switches are annotated as Q_{He} and Q_{Le}. Here, Q_{He} denotes the high-side switch of the eth electrode output (where e=1,2,3, . . . . E-1, E etc.), and similarly, Q_{Le} denotes the low-side switch of the eth electrode output. The semiconductor switches may be metal oxide semiconductor field effect transistors (MOSFETs) or insulated gate bipolar transistors (IGBTs) or maybe some combination of MOSFETs and IGBTs. For instance, Q_{He} could comprise 3 series-connected MOSFETs etc. It should be recognized that the topology of the pulse generator 104 depicted in FIG. 1 is merely an example and other pulse generator designs may be utilized.

An input voltage to the pulse-generator 104 is denoted as v_{bus}. As depicted in FIG. 1, this input voltage, v_{bus}, can be connected to different electrical sources by different switch circuits 106. The electrical sources input to the pulse generator 104 may be any of the N energy storage banks (which may be implemented by capacitor banks denoted as C_{B1}, C_{B2}, . . . C_{BN}), where each of these energy storage banks are charged from a respective charging power supply denoted PSU₁, PSU₂, . . . . PSU_{N}. In operation, if switch circuit 1 is turned on, while the other switch circuits 106 are turned off, the input voltage to the pulse generator 104 is powered from the charging power supply PSU₁ and the energy storage bank CB₁.

Similarly, the input voltage to the pulse generator 104 may be powered from a tissue-impedance measurement circuit 108 by turning on an Mth series-switch circuit and turning off all other series switch circuits. The tissue-impedance measurement circuit 108 may apply a low-voltage (e.g., less than 3V) sinusoidal output to the pulse-generator circuits input, which enables the measurement of the electrical impedance seen between an anode electrode and a cathode electrode of the pulse-generator circuit 104.

As shown, each of the switch circuits 106 includes semiconductor switches Q_{SFn} and Q_{SRn} where Q_{SFn} denotes a forward-blocking semiconductor switch of an nth series switch circuit. And similarly, the semiconductor switch Q_{SRn} denotes a reverse-blocking semiconductor switch of the nth series-switch circuit.

In many embodiments, each switch circuit 106 is bidirectional. Specifically, if both Q_{SFn} and Q_{SRn} are turned on, the series-switch circuit 106 allows current to commute in a forward and a reverse direction. Each switch circuit 106 can also be controlled to only allow current flow in the reverse direction by turning Q_{SFn} off and Q_{SRn} on. Each switch circuit 106 can also block current from flowing in both the positive and reverse directions by turning both switches Q_{SFn} and Q_{SRn} off.

This architecture allows the power supply 102 to operate as a PEF generator with a total of E electrode outputs (where E is two or more) powered from a total of N energy storage banks (where N is one or more), while also enabling a tissue-impedance measurement across any anode-cathode combination of electrode outputs.

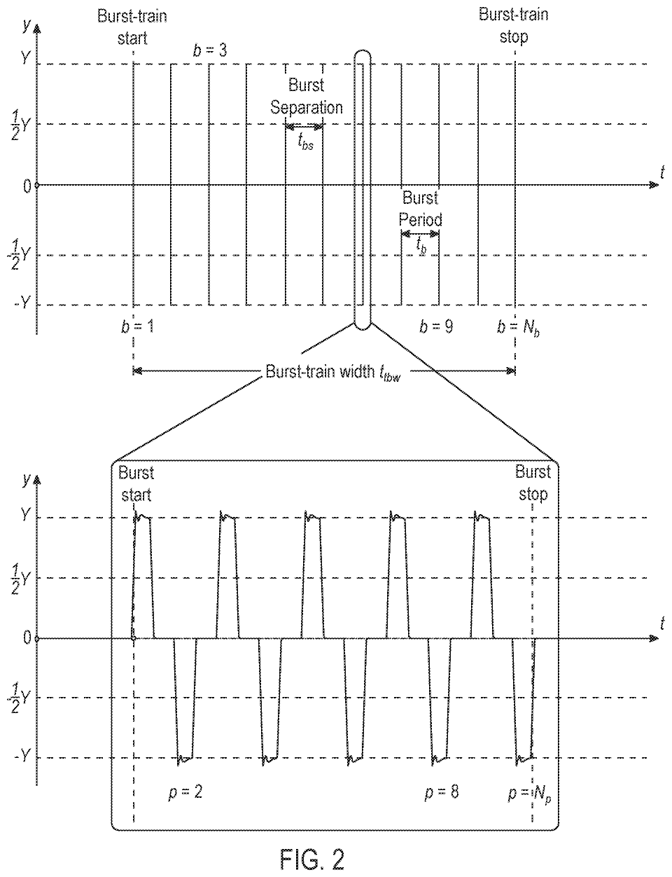

Referring next to FIG. 2, when the power supply 102 is operating as a PEF generator, the PEF generator can output a pulsed rectangular voltage across an anode and cathode electrode output terminals. As shown, the pulsed output voltage may be a finite burst-train waveshape, where each burst is a finite pulse train. Each burst of the burst train may be either a finite bipolar pulse train or a finite unipolar pulse train, or any other type of rectangular pulsed output shape. As discussed further herein, the burst-train waveshape may be used in medical treatments to induce electroporation in cells. In FIG. 2, an example burst-train waveshape is denoted y. The upper image depicts the burst train y(t) over a time epoch large enough to show the full burst-train waveshape from the first burst at b=1 to the Nb burst at b=Nb. The lower image zooms to show y(τ) on a shorter time epoch to show only a single burst, which in this case comprises of a finite bipolar pulse-train waveshape of Nₚ pulses.

PEF therapy may be used in cancer treatments to induce the biological phenomenon of reversible (RE) or irreversible (IRE) electroporation. Electroporation is a biological phenomenon in which high intensity, short duration electric field (Ē-field) pulses are applied to cells. Ē-field pulses with magnitudes of less than about 1 kV/cm induce the formation of small pores in the cell membrane. The cell membrane acts as a protective barrier to keep toxic substances outside the cell, which can be a disadvantage in medical treatment in that the cell membrane acts as an impermeable barrier to many drugs. With application of RE electroporation, pores form in the cell membrane to cause the membrane to become permeable to many drugs that otherwise could not cross the barrier.

In the medical field, PEF generators are of particular use in cancer therapy where RE electroporation can be applied to tumors in combination with previously impermeable anti-cancer drugs, to enable the drugs to cross the cell membrane and induce cell and tumor death. When Ē-fields higher than about 1 kV/cm are applied to cells, IRE electroporation can be induced. In this case the applied Ē-fields are strong enough to damage the cell membrane beyond repair, thus inducing cell death without application of any drugs. Thus, IRE electroporation may be used in medical treatments that require ablation of human tissue. A common example is the use of PEF in pulsed-field ablation (PFA) therapy for cardiac arrhythmia or in treating skin lesions.

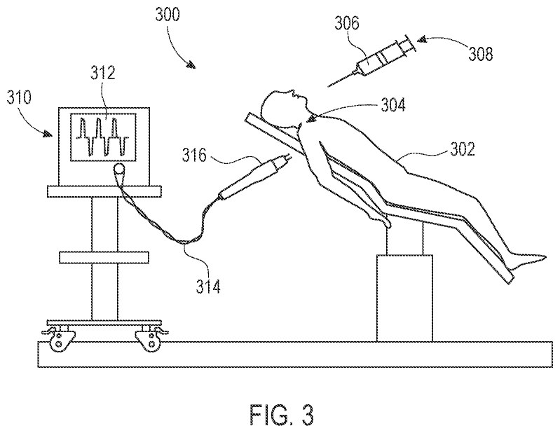

The use of a PEF generator to effect electroporation of cancer cells in combination with application of anti-cancer drugs to the cancer cells is sometimes referred to as electrochemotherapy (ECT). ECT is useful for treating skin tumors that are unsuitable for treatment by other methods such as resection. A typical patient setting 300 for ECT treatment is shown in FIG. 3 for a patient 302 with a skin tumor 304. A clinician delivers an anti-cancer drug 306 via an injection from syringe 308 into tumor 304, or in some cases intravenously. Electroporator 310 is then used to generate high intensity, short duration Ē-field pulses. PEF generator 312 is a subcomponent of electroporator 310 that produces the Ē-field pulses. Electroporator 310 may also include a user interface (UI) such as a touch screen and a foot pedal or other mechanism for controlling pulse delivery. Pulses are delivered from PEF generator 312 to patient 302 via cable 314 and probe 316. Probe 316 acts as a mechanical interface between PEF generator 312 and patient 302 and may be, for example, a hand-held probe in a plastic housing. Steel needles are configured on the tip of probe 316 to penetrate tumor 104. The pulsed output from PEF generator 312 is applied across these needles to induce an Ē-field within tumor 104 for RE electroporation.

As with any medical treatment, the safety of the patient is of utmost importance. Since the output of the PEF generator is applied directly to human tissue, patient safety and protection must be significant considerations in the generator design. However, the PEF generator must also provide the high voltages (up to 3 kV), current (up to 50 A) and power levels (up to 150 kW) needed to induce electroporation. This creates a significant design challenge, because a generator that outputs such high-power levels could potentially generate dangerous outputs that are applied to the patient. Accordingly, one aspect of this disclosure is a firmware independent over-energy protection (OEP) circuit that limits the energy that can be supplied by the PEF generator, even in the case of a firmware failure such as a frozen microcontroller.

Again, while described for exemplary purposes in a medical context, this disclosure is not so limited, and there are many other applications and contexts in which it may be desirable or necessary to limit the energy that can be output by a PEF generator. In this regard, the OEP circuit of this disclosure is suitable for use with various PEF generators in a medical or non-medical context having different output voltages, currents, and pulse widths, and functions to limit the supplied energy to a level that is appropriate and safe for the application in which the PEF generator is operating. In an ECT application, for example, an output of 50 J or less is generally considered safe and not likely to cause thermal damage to human tissue.

In skin ECT, high voltage outputs on the order of about 400V are needed to create the strong Ē-fields needed for successful electroporation. High currents of up to 20 A can be induced when treating a large volume of tissue with strong E-fields. Because tumors vary in terms of volume, water content, and level of necrosis, the output current varies on a tumor-by-tumor basis. In general, the deeper the needles on probe 316 must penetrate the tumor, the more current will be drawn. Applying a 400V, 20 A pulse delivers a very high peak power level from the PEF generator of 8 kW. While delivering an 8 kW DC power level to a patient seems unsafe, this is the required instantaneous power needed to electroporate skin cancer cells in tumors. For this reason, PEF generators in an ECT application are generally configured to generate a pulsed output that delivers the high-peak power level to the tumor for only a very small window of time in a burst of energy. The PEF generator delivers no output power for the remainder of the time as it is recharging its internal energy storage capacitor banks.

The needles inserted into the patient are typically long and narrow, and as a result, it is possible for the needles to deflect on insertion into the targeted tissue. In the case where needles deflect inward it is possible that the needles connected to the PEF generator cathode contact the needles connected to the PEF generator anode. This causes a short-circuit condition across the needle outputs. There are of course other possible situations that give rise to a short-circuit condition. For example, the targeted tissue may have a very high conductivity, the target tissue may become increasingly conductive during the application of electroporation, or arcing may occur in the probe. It is also possible that a semiconductor switch within the pulse-generator may fail, which may create a short circuit. As a result, it is necessary for PEF generators to have robust short-circuit protection.

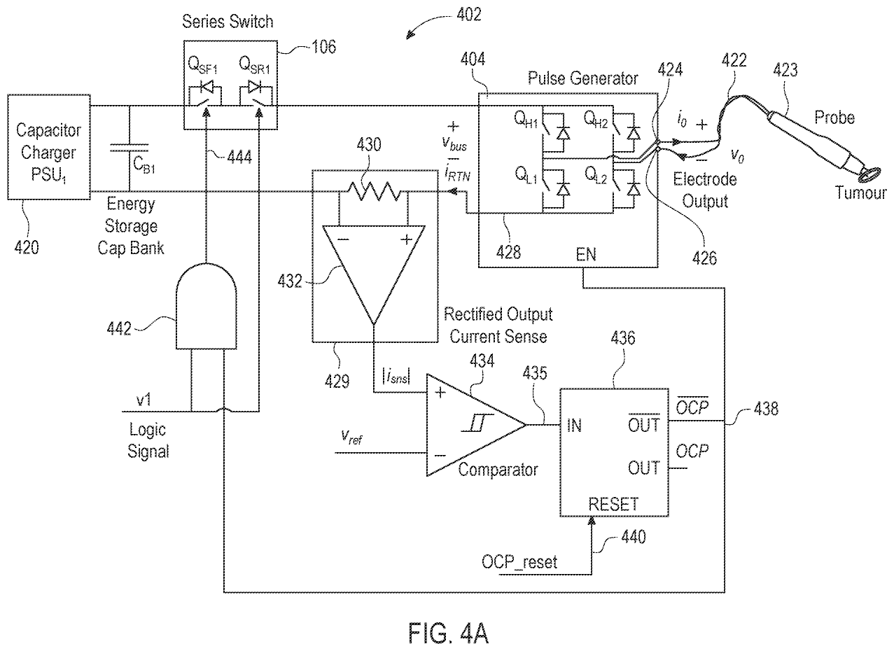

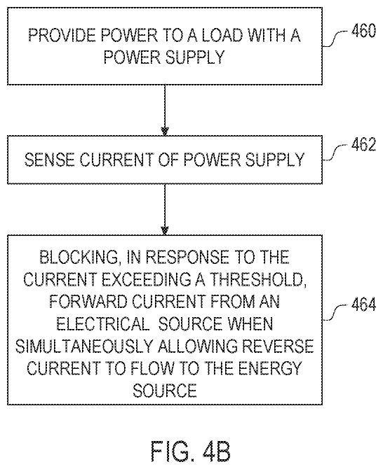

Referring next to FIG. 4A, shown is a block diagram illustrating how the overcurrent protection may be implemented. Here, a power supply 402 is similar to the power supply 102 of FIG. 1, but the circuitry is reduced for simplicity to include one energy storage bank, C_{B1}, and a pulse generator 404 with two electrode outputs, but the architecture described with reference to FIG. 4A is representative of architectural extensions such as the architecture depicted in FIG. 1, which includes multiple energy storage banks and more than two electrode outputs. In FIG. 4A, a capacitor charger power supply unit (PSU) 420 is used to charge a capacitor bank denoted as C_{B1}.

As shown, the pulse generator 404 includes a switch network comprising an H-bridge circuit comprising switches Q_{H1}, Q_{L1}, Q_{H2}, and Q_{L2}, which collectively operate to receive power from the energy storage bank, C_{B1}, and provide pulses between a first node 424 and a second node 426 to produce the pulsed output voltage, vo. In one example application, a twisted-pair cable 422 of a probe 423 is coupled between the first node 424 and the second node 426, and the pulsed output voltage, vₒ, is applied to the twisted-pair cable 422 and probe 422, which enables the output voltage, vₒ, to be applied across a load (e.g., a tumor volume), which induces a pulsed output current, io, to flow in the load (e.g., tumor 304). As previously described, medical electroporation is an example application and other applications are certainly contemplated.

As shown, a switch control signal, Q_{SI}, is provided to both an input of an AND gate 442 and the reverse-blocking semiconductor switch, Q_{SR1}. Another input to the AND gate 442 is the inverted OCP signal 438, and an output of the AND gate 442 is provided to the forward-blocking semiconductor switch, Q_{SF1}. While referring to FIG. 4A, simultaneous reference is made to FIG. 4B, which is a flowchart depicting a method that may be traversed in connection with embodiments disclosed herein.

In operation, when power is provided by the power supply 402 (Block 460), the over-current protection aspects of the power supply 402 work, in part, by sensing the current of the power supply (Block 462). For example, current, i_{RTN}, on a return leg 428 of the power supply 402 may be sensed. This current, i_{RTN}, represents the rectified version of the output current, iₒ, and this current, I_{RTN}, may be sensed using a current-sense circuit 429 that may comprise a shunt resistor 430 and an operational amplifier 432. The output of the current-sense circuit 429 is a voltage signal denoted |isns|, and this voltage signal represents a scaled and rectified version of the output current, iₒ. The voltage signal (also referred to as a rectified-sense current signal) |isns|, is compared, using comparator 434, to a reference voltage, v_{ref}. This reference voltage, v_{ref}, establishes a threshold for the current, and v_{ref} may be a constant voltage reference or may be an adjustable reference provided, for example, by an output of a digital-to-analog (DAC) converter. Because there is a direct relation between the voltage signal, |isns| and the output current, iₒ, the reference voltage, v_{ref}, can operate as a threshold for the output current, iₒ.

In response to the sensed current exceeding the threshold, forward current from the electrical source (e.g., the capacitor charger 420 and energy storage bank C_{B1}) is blocked while reverse current is simultaneously allowed to flow to the energy source (Block 464). In the embodiment of FIG. 4A, when the rectified-sense current signal |isns| exceeds the value of the reference voltage, v_{ref}, an output 435 of the comparator 434 is triggered high, this output 435 is latched to a high state using a latch circuit 436. The output of the latch circuit 436 is a logic signal denoted OCP, which represents if an over current event has occurred. The latch circuit 436 also has an inverted output denoted by an inverted OCP signal 438. In the event of an over current event, the inverted OCP signal 438 (provided to an enable, EN, input of the pulse generator 404) is pulled low, which disables the pulse generator 404. In addition, the inverted OCP signal 438 also stops (as discussed further herein) the switch circuit 106 from conducting in the forward direction only. It is noted that the switch circuit 106 is controlled to block forward current to prevent any more energy from being released from the capacitor bank, C_{B1}, while simultaneously allowing reverse current to flow to allow any stored inductive energy in the output cable 422 to return to the energy storage bank, C_{B1}. The energy stored in the output cable 422 is much less than the energy stored in the energy storage bank, C_{B1}; thus, allowing the switch circuit 106 to conduct in the reverse direction during an over current event, which effectively clamps the bus voltage, v_{bus}, to a voltage of the energy storage bank, C_{B1}. If the switch circuit 106 was commanded to block the flow of current in the reverse direction, the stored inductive energy in the cable 422 can cause a voltage overshoot of v_{bus} (at an input of the pulse generator 404), which could potentially damage the switches Q_{H1}, Q_{L1}, Q_{H2}, and Q_{L2} in the pulse generator 404.

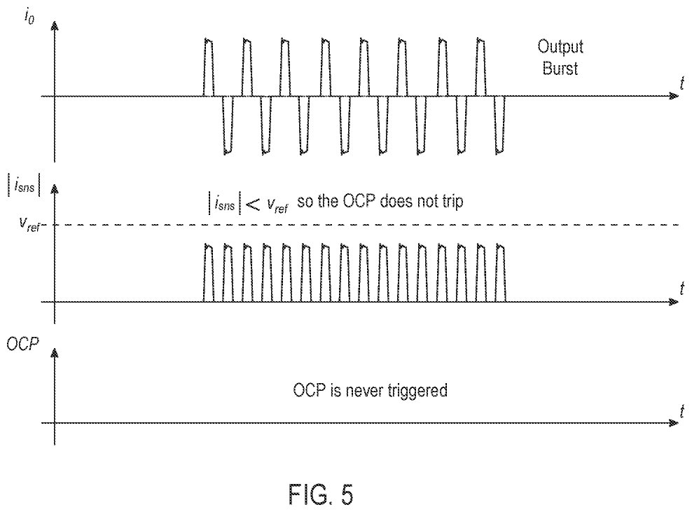

During normal operation, the switch control signal, Q_{SI}, and the inverted OCP signal 438 are both high, so an output 444 of the AND gate 442 is high, and as a consequence, both the forward-blocking semiconductor switch, Q_{SF1} and the reverse-blocking semiconductor switch, Q_{SR1}, are ON (positioned closed), which enables forward current to flow through the switch circuitry 106 to the pulse generator 404, and the pulse generator 404 may output a typical rectangular pulsed output voltage. This typical pulsed output voltage is depicted in FIG. 5. So long as the output current is not high enough to cause |isns| to exceed v_{ref}, then the OCP event is not triggered, and the pulse generator 404 and switch circuit 106 are enabled.

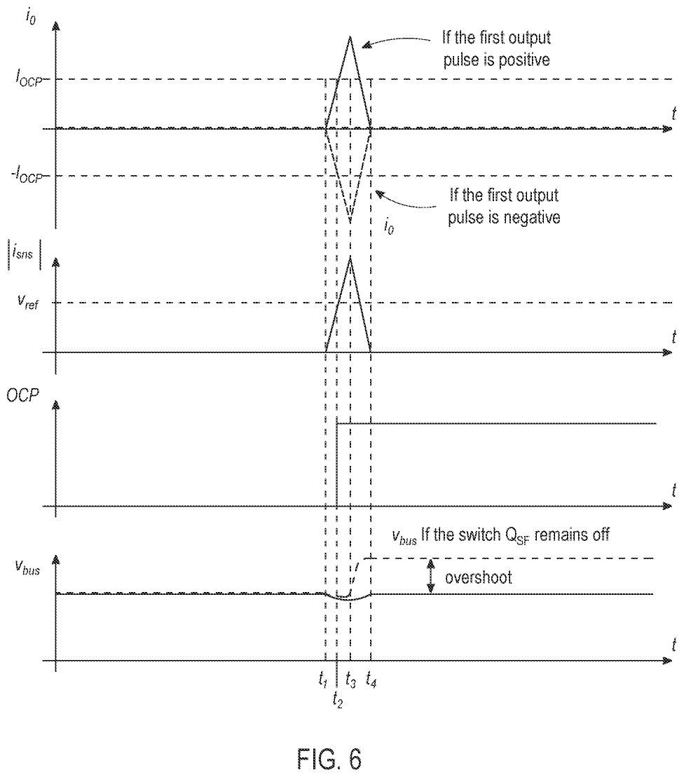

If the output of the power supply 402 is short-circuited (across the first node 424 and the second node 426), during the first pulse of the pulse-train output, an excessively high current will flow in the output cable 422. This is depicted in FIG. 6. From the time interval t1<t<t2, the output current, iₒ, of the power supply 402 begins to rise. The di/dt rising slope of this output current, iₒ, is limited by parasitic inductance of the power supply 402 and the switching speed of the switches Q_{H1}, Q_{L1}, Q_{H2}, and Q_{L2}. At the time instance t2, the output current exceeds a trip level denoted I_{OCP}, similarly at this trip level, the rectified current-sense signal |isns| exceeds the threshold established by reference voltage v_{ref}, which in turn, causes the output, OCP, of the latch 436 to trigger high and the inverted OCP signal 438 to trigger low. The pulse generator 404 and the series switch 106 have a delay before they stop conducting, so for the time interval t2<t<t3, the forward-blocking semiconductor switch, Q_{SF1} and the reverse-blocking semiconductor switch, Q_{SR1}, are ON (positioned closed) as during normal operation and the output current continues to rise.

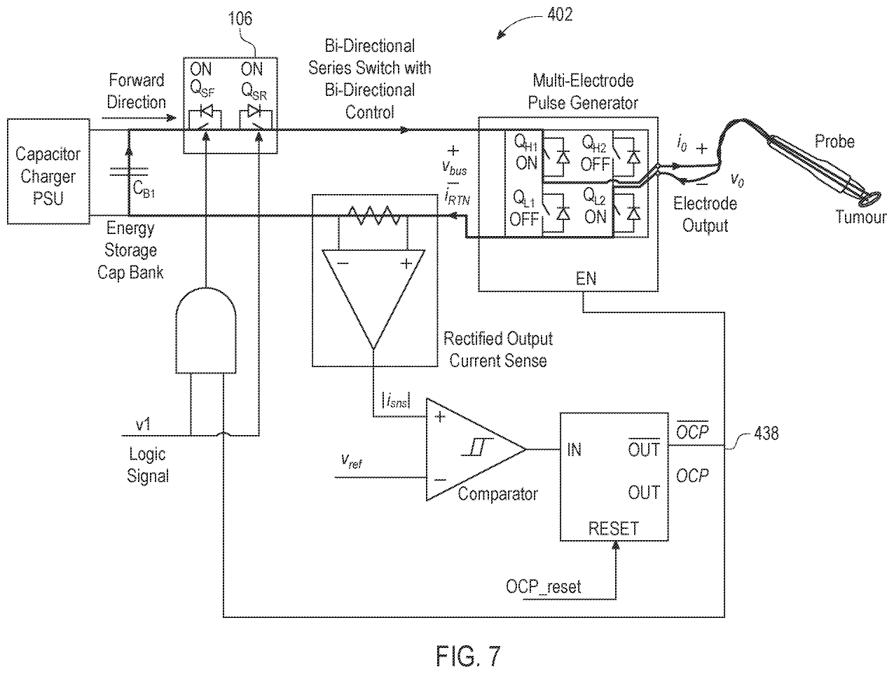

The current path during the time interval t1<t<t3, is depicted in FIG. 7. As the output current rises, the current flows from the capacitor bank, C_{B1}, in the forward direction through the switch circuit 106, and through switches Q_{H1} and Q_{L2} of the pulse generator 404.

At the time instance t3, there can be a very high instantaneous current (e.g., in excess of 100 A), flowing though the output cable 422, so there can be significant stored inductive energy in the output cable 422 at this instance. At the time instance t3, the latch 436 places the inverted OCP signal 438 low, which triggers all switches (Q_{H1}, Q_{L1}, Q_{H2}, and Q_{L2}) in the pulse generator 404 off (open). In addition, because the inverted OCP signal 438 is low, the output 444 of the AND gate 442 is low; thus, triggering the forward-blocking semiconductor switch, Q_{SF1}, OFF (open) while the reverse-blocking semiconductor switch, Q_{SR1}, remains ON (positioned closed) so that the switch circuit 106 is off in the blocking direction only.

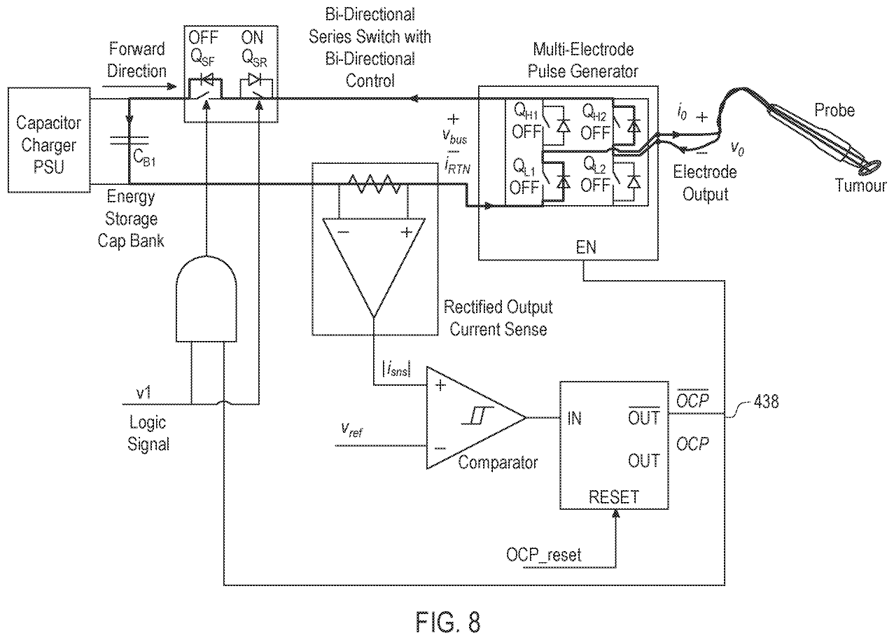

For simplicity, it is assumed these devices transition instantaneously to the off state and all inductive energy in the cable 422 continues to flow through the body diodes (of the switches Q_{H1}, Q_{L1}, Q_{H2}, and Q_{L2}) of the pulse generator 404 as shown in FIG. 8. In reality, the devices do not turn off instantaneously, and some of the inductive energy in the cable 422 will be dissipated as switching losses in the switches Q_{H1}, Q_{L1}, Q_{H2}, and Q_{L2} of the pulse generator 404 during device turn off. The remaining inductive energy then conducts through the body diodes of the pulse generator 404. The inductive energy causes a reverse current to flow through the switch circuit 106, so the series switch 106 is controlled to keep the reverse-blocking semiconductor switch, Q_{SR1}, ON (positioned closed), allowing this reverse current to flow. This behavior also clamps the bus voltage, v_{bus}, at the voltage of the energy storage bank, C_{B1}; thus, preventing an overshoot on v_{bus}. It is also noted that the switch circuit 106 is turned off in the forward direction by turning Q_{SF} OFF (open) because this will prevent the energy storage bank, C_{B1}, from being shorted in the case of a half-bridge in the pulse generator 404 has failed due to a short.

If during an OCP event the switch circuit 106 is turned off to block both forward and reverse current, the inductive energy stored in the output cable will still flow back into the input of the pulse generator 404 and cause a significant voltage overshoot across the voltage, v_{bus}, which is also depicted in FIG. 8. As a consequence, switch circuit 106 allows reverse current to flow during an OCP event.

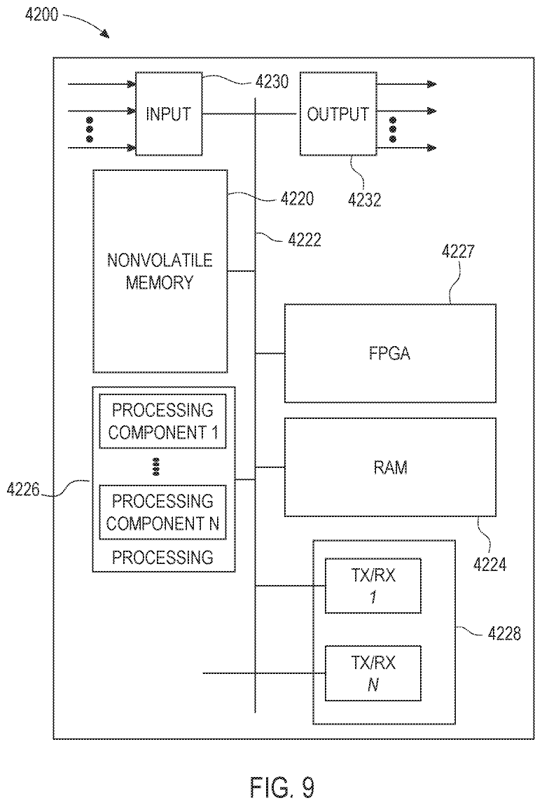

Beneficially, the over current protection (OCP) circuitry depicted in FIGS. 4A, 7, and 8, is firmware independent to latch the pulse generator 404 off and latch the switch circuit 106 off in the forward conduction direction. But the OCP circuitry may operate in connection with other hardware, in processor-executable code encoded in a non-transitory tangible processor readable storage medium, or in a combination of the two. Referring to FIG. 9 for example, shown is a block diagram 4200 depicting physical components that may be utilized, for example, as a controller in power supply 102 and/or devices incorporating power supply 102, such as electroporator 310.

As shown, bus 4222 is coupled to nonvolatile memory 4220, random access memory (“RAM”) 4224, processing portion 4226 that includes N processing components, field programmable gate array (FPGA) 4227, and transceiver component 4228 that includes N transceivers. None of these components are required, and any combination of these may be included in the systems disclosed herein. For instance, where FPGA 4227 is implemented, processing portion 4226 may not be used, and vice versa. Although the components depicted in FIG. 9 represent physical components, FIG. 9 is not intended to be a detailed hardware diagram; thus, many of the components depicted in FIG. 9 may be realized by common constructs or distributed among additional physical components. Moreover, it is contemplated that other existing and yet-to-be developed physical components and architectures may be utilized to implement the functional components described with reference to FIG. 9.

In general, nonvolatile memory 4220 is non-transitory memory that functions to store (e.g., persistently store) data and processor-executable code (including executable code that is associated with effectuating the methods described herein). In some embodiments, nonvolatile memory 4220 includes bootloader code, operating system code, file system code, and non-transitory processor-executable code to facilitate the execution of a method to coordinate operation of power supply 300 as described herein.

In many implementations, nonvolatile memory 4220 is realized by flash memory (e.g., NAND or ONENAND memory), but it is contemplated that other memory types may be utilized as well. Although it may be possible to execute the code from nonvolatile memory 4220, the executable code in the nonvolatile memory is typically loaded into RAM 4224 and executed by one or more of the N processing components in processing portion 4226.

The N processing components in connection with RAM 4224 generally operate to execute the instructions stored in nonvolatile memory 4220 to enable a method for operating power supply 300 and devices incorporating power supply 300 such as electroporator 110. For example, non-transitory, processor-executable code to effectuate the methods described herein may be persistently stored in nonvolatile memory 4220 and executed by the N processing components in connection with RAM 4224. As one of ordinarily skill in the art will appreciate, processing portion 4226 may include a video processor, digital signal processor (DSP), micro-controller, graphics processing unit (GPU), or other hardware processing components or combinations of hardware and software processing components (e.g., an FPGA or an FPGA including digital logic processing portions).

In general, input component 4230 generally operates to receive one or more analog and/or digital signals (e.g., current and/or voltage signals) and output component 4232 generally operates to provide one or more analog or digital signals. For example, the output component 4232 may produce the voltage Vref (representing the current threshold), and non-transitory, processor-executable code may be used to enable an operator of power supply 300 to configure the threshold. It is also contemplated that a display may be incorporated with the components of the power supplies disclosed herein to provide information about the current delivered during operation. The output component 4230 may also provide the switch control signal, Q_{SI}, and the OCP_reset signal.

Transceiver component 4228 includes N transceiver chains, which may be used for communicating with external devices via wireless or wireline networks. Each of the N transceiver chains may represent a transceiver associated with a particular communication scheme (e.g., WiFi, Ethernet, Profibus, etc.).

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The word “exemplary” as used means “serving as an example, instance, or illustration.” Any embodiment described herein as “exemplary” should not be construed as preferred or advantageous over other embodiments.

The flowcharts and block diagrams in the drawing figures illustrate the architecture, functionality, and operation of possible implementations of systems, methods, and computer program products according to various embodiments of this disclosure. In this regard, some blocks in the flowcharts and block diagrams may represent a module, segment, or portion of code, which comprises one or more executable instructions for implementing the specified logical function(s). In some implementations, the functions noted in the block may occur out of the order set forth in the drawings. For example, two blocks shown in succession may, in fact, be executed substantially concurrently, or be executed in the reverse order, depending upon the functionality involved. It will also be understood that each block and combinations of blocks in the flowcharts and block diagrams can be implemented by special purpose hardware-based systems that perform the specified functions or acts, or combinations of special purpose hardware and computer instructions.

Although the terms first, second, third etc. may be used herein to describe various elements, components, regions, layers and/or sections, it will be understood that these elements, components, regions, layers and/or sections are not limited by these terms. These terms are only used to distinguish one element, component, region, layer or section from another region, layer, or section. Thus, a first element, component, region, layer, or section discussed below could be termed a second element, component, region, layer, or section without departing from the teachings of this disclosure.

The terminology used herein is for the purpose of describing particular embodiments only and is not intended to limit this disclosure. As used herein, the singular forms “a,” “an,”and “the” are intended to include the plural forms as well, unless the context clearly indicates otherwise. The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes all combinations of one or more of the associated listed items and may be abbreviated as “/”.

As used herein, the recitation of “at least one of A, B and C” or “at least one of A, B or C” is intended to mean “either A, B, C or any combination of A, B and C.” This description is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the scope of this disclosure is not limited to the embodiments shown herein but is to be accorded the widest scope consistent with the principles and novel features disclosed herein.

1. A power supply comprising:
a pulse generator configured to receive power from an electrical source and provide power between a first node and a second node;
a switch circuit coupled to the pulse generator;
a sensor configured to sense current of the power supply and to generate a signal indicative of the current; and
control logic configured, in response to the signal exceeding a threshold, to control the switch circuit to block forward current from the electrical source while simultaneously allowing reverse current to flow to the electrical source.

2. The power supply of claim 1, wherein the electrical source is a capacitor bank, and the power supply further comprises a capacitor charger.

3. The power supply of claim 1, wherein the switch circuit comprises a forward-blocking semiconductor switch and a reverse-blocking semiconductor switch, the forward-blocking semiconductor switch arranged in series with the reverse-blocking semiconductor switch.

4. The power supply of claim 3, wherein the control logic includes:
a comparator configured to provide an output signal if the signal exceeds a reference signal; and
a latch circuit configured to provide a latched signal in response to receiving the output signal from the comparator, wherein the latched signal triggers the forward-blocking semiconductor switch to close.

5. The power supply of claim 4, wherein the control logic further comprises:
an AND gate to provide a control signal for the forward-blocking semiconductor switch responsive to the latched signal and a switch control signal.

6. The power supply of claim 1, wherein the switch circuit comprises a bidirectional switch, and wherein the control logic comprises a firmware-independent latch to latch the pulse generator off and latch the bidirectional switch off in a forward conduction direction to block the forward current.

7. The power supply of claim 6, wherein the wherein the switch circuit comprises a forward-blocking semiconductor switch and a reverse-blocking semiconductor switch, the forward-blocking semiconductor switch is arranged in series with the reverse-blocking semiconductor switch, and the control logic is configured to:
turn both the forward-blocking semiconductor switch and the reverse-blocking semiconductor switch on to allow current to flow in a forward and a reverse direction;
turn the forward-blocking semiconductor switch off to allow current to flow in the reverse direction only; and
turn both the forward-blocking semiconductor switch and the reverse-blocking semiconductor switch off to block current from flowing in both the forward and reverse directions.

8. The power supply of claim 1, wherein the generator is configured to apply power pulses between the first node and the second node.

9. The power supply of claim 1, wherein the sensor comprises a shunt resistor and an operational amplifier, the shunt resistor being configured to produce a voltage drop proportional to the current, and the operational amplifier being configured to produce the signal indicative of the current.

10. The power supply of claim 1 comprising a probe detachably coupled to the first node and the second node and configured with a first needle and a second needle to enable the pulses to be applied to a patient.

11. A method comprising:
providing power to a load with a power supply;
sensing current of the power supply; and
blocking, in response to the current exceeding a threshold, forward current from an electrical source while simultaneously allowing reverse current to flow to the electrical source.

12. The method of claim 11, comprising:
providing the power to the load from a capacitor bank.

13. The method of claim 11, wherein the blocking comprises blocking the forward current with a bidirectional switch triggered by a firmware-independent latch to latch the bidirectional switch off in a forward conduction direction to block the forward current.

14. The method of claim 13, wherein the blocking comprises blocking a drain-source current path of a forward-blocking semiconductor switch while the allowing comprises allowing the reverse current to flow through a body diode of the forward-blocking semiconductor switch.

15. The method of claim 11, wherein providing power comprises providing pulsed power to a patient via a probe.

16. The power supply of claim 1, wherein the pulse generator comprises a switch network.

17. The power supply of claim 16, wherein the switch network comprises an H-bridge circuit.

18. The power supply of claim 1, wherein the pulse generator comprises a multi-electrode pulse generator.

19. The method of claim 11, wherein sensing current comprises sensing current with a shunt resistor and an operational amplifier.

20. The method of claim 15, wherein providing power comprises providing pulsed power to a patient via a multi-electrode probe.
